(19) United States
(12) Patent Application Publication
(10) Pub. No.: US 2010/0228145 A1
(43) Pub. Date: Sep. 9, 2010

(54) DEVICE AND METHOD FOR EVALUATING SYMPATHETIC FUNCTION USING ELECTROOCULOGRAPHY

(76) Inventors: Bo-Jau KUO, Taipei City (TW); Ching-Hsiu YANG, Taipei City (TW)

Correspondence Address: Muncy, Geissler, Olds & Lowe, PLLC, 4000 Legato Road, Suite 310, FAIRFAX, VA, 22033, US

(21) Appl. No.: 12/399,678

(22) Filed: Mar. 6, 2009

(51) Int. Cl. A61B 5/05

(52) U.S. Cl. 600/558

(57) ABSTRACT

The present invention provides a device and method for evaluating sympathetic function using electrooculography having advantage of simply analytical process and portability. The device for evaluating sympathetic function using electrooculography mainly comprises a plurality of electrode pastes and an electrooculography detector. Moreover, the structure of the device is further selected from the group consisting of necklace, hat block, adhesive bandages, and button. In practice, the device and method for evaluating sympathetic function using electrooculography according to the present invention utilizes a technology of micro physiological wireless transmitter and sensing synchronizing with analysis to be a simple and precise detective tool of detecting sympathetic function with electro-oculogram (EOG).

### BACKGROUND OF THE INVENTION

1. Field of the invention

The present invention generally relates to a device and method for evaluating sympathetic function using electrooculography, and more particularly to convenient and precisely-analyzing device and method for evaluating sympathetic function using electrooculography, designed to be used in long term estimation of cardiovascular disease, career, and sleep disorder of health or patients.

2. Description of the Related Art

Human autonomic nervous system controls the physiological function relevant to life maintenance through the whole body, which comprises blood pressure, heart rate, tracheal resistance, perspiration, body temperature, and metabolism. These nerve operations can process automatically. Autonomic nervous system mainly includes sympathetic nerves and parasympathetic nerves. Generally speaking, the former is related to resist the environment, whereas the latter is related to propagation. For example, when a person is excited, the former will increase the blood pressure and dilate the pupils while the latter will cause gastrointestinal secretion and genital erection. In general, sympathetic functions and parasympathetic functions are active in young persons, but rather inactive in old persons; in males, sympathetic functions prevail but parasympathetic functions yield; conversely, parasympathetic functions excel sympathetic functions in females. So we can know that sympathetic nerves and parasympathetic nerves are closely related to the daily operation of a human body. Autonomic imbalance may induce various acute and chronic diseases, for example, heart disease, hypertension, etc., and may even lead to a sudden death, if serious. Hence, the protection for autonomic nervous system is not only an important issue in medicine but also a personal concern to an individual everyday. If we can control the function of autonomic nervous system efficiently, such as changing daily schedule, regulating respiratory frequency, and even processing the movement, relaxation, and reducing movement of the eyes, the diseases can be prevented or improved. Therefore, it will be an important regimen.

The strong and weak of the sympathetic function are not only related to diseases, but also related to the variation of sleep, and alertness and concentration while awake. Detecting sympathetic function efficiently is helpful to improve the alertness and concentration while awake, or to decrease the alertness and concentration before sleep in order to promote falling asleep. In addition, controlling sympathetic function is also a common used method in psychology. However, the traditional measurements of sympathetic function consume time and inaccurate, for example, the subject should stop breathing or lie on a stand-up bed so that the change of blood pressure caused by stopping breathing or stand-up bed can be detected to recognize the sympathetic function. Recently, there are some harmless measurements such as heart rate variation analysis, but a convenient and precisely-analyzing tool detecting sympathetic function is not present in the business situations. Hence, the examination of sympathetic function is still used in hospital or research instead of in personal life.

In recent years, plenty of new technologies to evaluate the autonomic functions were successfully developed. Given the sophisticated computer hardware and software know-how available, today it is possible to detect and perform quantitative analysis of a person autonomic cardiac activity in light of the minute fluctuations of hear rate, known as heart rate variability (HRV), taken while the person is at rest. In other words, the new technologies allow a user to analyze or evaluate a normal person's autonomic functions without interfering with the person's daily life. Researchers discovered that the minute fluctuations of heart rate variability, which can be represented by total power (TP), can be definitely divided into two groups, that is, high-frequency (HF) component and low frequency (LF) component. The HF component is synchronous to animals' breath signals, so it is also known as breathing component. The source of the LF component is relevant to vascular motion or baroreflex.

Many physiologists and cardiologists believe that the HF component or total power reflects parasympathetic functions, whereas the ratio of LF component to HF component (LF/HF) reflects sympathetic activity. For instance, patients diagnosed with intracranial hypertension usually have relatively low heart rate variability. The public health investigation of American Framingham found that the death rate of an elder whose LF component of heart rate variability decreases by a standard deviation is 1.7 times that of normal person. Nowadays, a series of software and hardware, which can process spectrum analysis directed against various physiological signals on-line, have been developed. If the LF components of heart rate and blood pressure are the index of depth of anesthesia, for example, it can be found in the intensive care unit that the survival rates of patients decrease while heart rate variability decreases and the LF component of heart rate variability vanishes in a brain-dead person. Furthermore, there are changes in heart rate variability in a patient who exhibits rejection reactions after heart transplantation.

Electrooculography (EOG) is a basis of detecting physiological condition, and is sometimes used for record. The records of eye movement are mainly based on the potential change between cornea and retina. In fact, there is a minute potential change existing between cornea and retina, so that eye balls in a head are the same as an electric field in a conductor. When the eye balls move rapidly or slowly, there will be a bigger potential change produced between cornea and retina. The potential change of eye movement will be recorded because the positions of eye movement electrodes are fixed. When eye balls move rapidly or slowly, the recorded potential of the electrode near the cornea will be positive while that of the electrode near the retina will be negative. Therefore, the two recorded spectrum will be present an opposite shift.

At present, EOG signal is mostly applied in sleep check which is related to the determination of rapid eye movement. Furthermore, EOG is often applied to control brain-computer interface system or in some psychotherapy. According to the above problems, a device and method for evaluating sympathetic function using electrooculography is reported, which the sympathetic function of a subject is expected to decrease or increase efficiently by improving the EOG signal of the subject.

### BRIEF SUMMARY OF THE INVENTION

The primary objective of the present invention is to provide a device for evaluating sympathetic function using electrooculography, with a view to simplifying the analytical process and carrying out automation, to be a sympathetic function examining tool.

The another objective of the present invention is to provide a method for evaluating sympathetic function using electrooculography, by which the analytical process can be simplified, to be a sympathetic function examining technology of autonomic nervous system.

The another objective of the present invention is to provide a system for evaluating sympathetic function using electrooculography, by which the estimation of cardiovascular disease, career, and sleep disorder of healthy people or various patients can be supplied.

To achieve the first objective, the present invention provides a device for evaluating sympathetic function using electrooculography which mainly comprises a plurality of electrode pastes and an eye movement detector. The plurality of electrode pastes are placed at the tail of each eye, and the mastoid or earlobe of the left ear and right ear of a person, whereas the eye movement detector is used for capturing an eye movement signal of the person and converting it into an electrooculography.

According to one aspect of the present invention of a device for evaluating sympathetic function using electrooculography, the structure of the device is further selected from the group consisting of necklace, hat block, adhesive bandages, and button.

To achieve the second objective, the present invention provides a method for evaluating sympathetic function using electrooculography which mainly comprises the following steps of capturing an eye movement signal of a person, performing an analog-to-digital conversion of the eye movement signal after amplifying, converting the eye movement signal into a electrooculography, and quantifying the electrooculography and setting up a threshold value as a basis of feedback controlling.

To achieve the third objective, the present invention provides a system for evaluating sympathetic function using electrooculography which mainly comprises an eye movement sensor, a micro-controller unit, and a feature extraction unit. The eye movement sensor is used for capturing an eye movement signal of the person and converting it into an electrooculography. The micro-controller unit is used for processing the electrooculography from the eye movement sensor. The feature extraction unit is used for analyzing the electrooculography from the micro-controller unit and sending it back to the micro-controller unit.

According to one aspect of the present invention of a system for evaluating sympathetic function using electrooculography, the system further comprises a switch unit used for deciding the analysis result of the electrooculography from the micro-controller unit and sending a deciding result back to the micro-controller unit.

By utilizing physiological signal detector and a technology of sensing synchronizing with analysis to detect and analyze the eye movement signal, a simple and precise detective tool of detecting sympathetic function can be realized. It can be used in the estimation of cardiovascular disease, career, and sleep disorder of health or patients.

The invention itself, though conceptually explained in above, can be best understood by referencing to the following description, taken in conjunction with the accompanying drawings.

### BRIEF DESCRIPTION OF THE DRAWINGS

All the objects, advantages, and novel features of the invention will become more apparent from the following detailed descriptions when taken in conjunction with the accompanying drawings.

FIG. 1 shows a schematic of the device for evaluating sympathetic function using electrooculography according to the present invention;

FIG. 2 shows a process flow chart of the system for evaluating sympathetic function using electrooculography according to the present invention;

FIG. 3 shows a process flow chart of the eye movement detector according to the present invention;

FIG. 4 shows a diagram of the eye movement frequency (0.05-0.5 Hz) intensity (PEOG) and the sympathetic function of heart (LF/HF) according to the present invention; and

FIG. 5 shows an analytical result of the eye movement frequency (0.05-0.5 Hz) intensity (PEOG) and the sympathetic function of heart (LF/HF) according to the present invention.

### DETAILED DESCRIPTION OF THE INVENTION

Although the invention has been explained in relation to several preferred embodiments, the accompanying drawings and the following detailed descriptions are the preferred embodiment of the present invention. It is to be understood that the following disclosed descriptions will be examples of present invention, and will not limit the present invention into the drawings and the special embodiment.

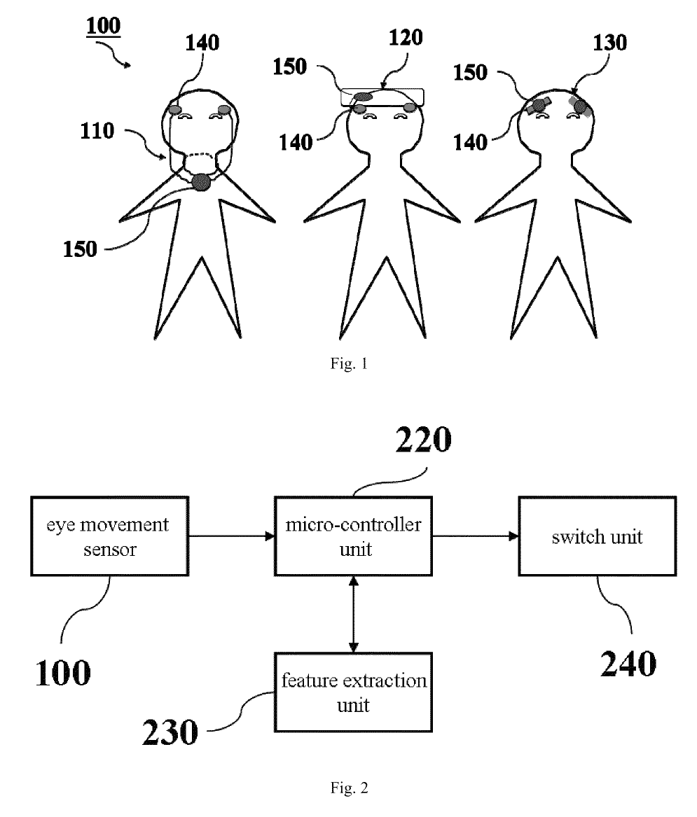

Referring to FIG. 1, it shows a schematic of the device 100 for evaluating sympathetic function using electrooculography according to the present invention. The device 100 for evaluating sympathetic function using electrooculography is an eye movement sensor which mainly comprises a plurality of electrode pastes 140 and an eye movement detector 150. The plurality of electrode pastes 140, which are one of reactive electrodes and the input electrodes of traditional electrocardiogram, are placed at the tail of each eye, and the mastoid or earlobe of the left ear and right ear of a person. The eye movement detector 150 is used for capturing an eye movement signal of the person and converting it into an electrooculography. After being amplified and filtered, these signals could be the sources of sympathetic function signals which are dealt with at following steps. The two electrode pastes 140, which are used for recording the eye movement, are usually placed at 1 cm above the tail of right eye and 1 cm below the tail of left eye respectively, while the reference electrodes are placed at the mastoid or earlobe of the left ear and right ear.

In accordance with the potential change of the eye balls, the front cornea represents positive potential while the back retina represents negative potential. When the eye balls of a subject are stationary, the diagram appearing on the eye movement spectrum mainly reflects brain wave signal. When the eye balls move up or right, a positive potential will be recorded and the recorded curve will shift downward, because the cornea of the right eye moves close to the right electrode. In the same condition, a negative potential will be recorded and the recorded curve will shift upward, because the retina of the left eye moves close to the left electrode. In the contrary, when the eye balls move down or left, a negative potential will be recorded and the recorded curve will shift upward, because the retina of the right eye moves close to the right electrode. In the same condition, a positive potential will be recorded and the recorded curve will shift downward, because the cornea of the left eye moves close to the left electrode. Moreover, the eyes will also move automatically when the subject winks the eyes, which produces the short change of potential, so that the two recorded spectrum will appear an opposite shift.

The device 100 for evaluating sympathetic function using electrooculography, which is also the structure of the eye movement sensor 100, is further selected from the group consisting of necklace 110, hat block 120, adhesive bandages 130, and button. The structure of eye movement sensor 100 combine with a tool that is easier to use, measure, and operate in personal life to become a measurement tool of sympathetic function of autonomic nervous system.

Based on the device disclosed in the present invention, a method for evaluating sympathetic function using electrooculography mainly comprises the following steps of capturing an eye movement signal of a person, performing an analog-to-digital conversion of the eye movement signal after amplifying, converting the eye movement signal into an electrooculography, and quantifying the electrooculography and setting up a threshold value as a basis of feedback controlling. Reducing eye movement is required when the feature of the electrooculography exceeds the threshold value, whereas increasing eye movement is required when the feature of the electrooculography is below the threshold value.

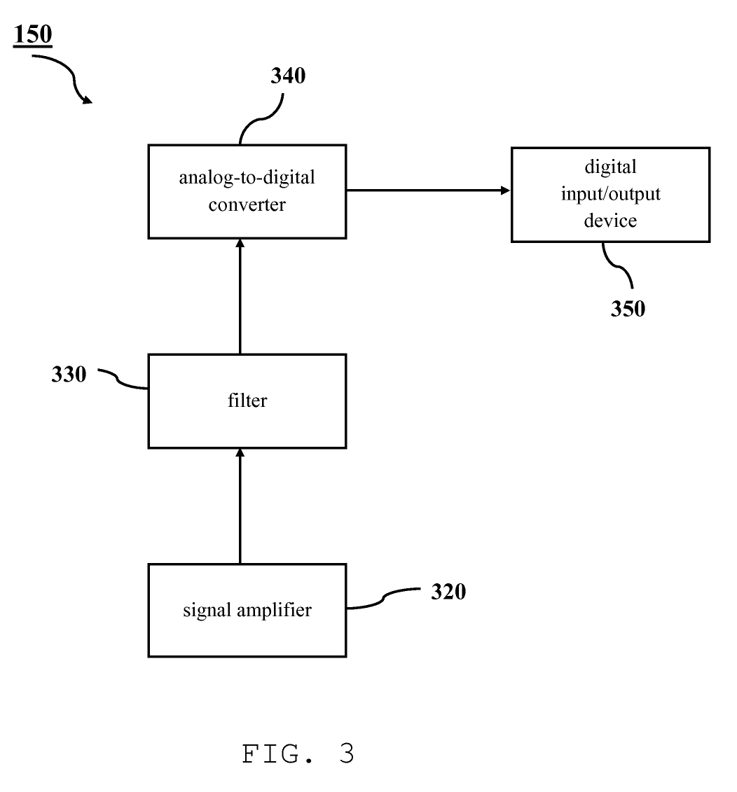

Referring to FIG. 2, it shows a process flow chart of the system for evaluating sympathetic function using electrooculography according to the present invention. The system mainly comprises an eye movement sensor 100, a micro-controller unit 220, a feature extraction unit 230, and a switch unit 240. It has to be noticed that the micro-controller unit 220, the feature extraction unit 230, and the switch unit 240 can be put in a circuit together with the eye movement sensor 100; they also can be put in the different circuit respectively. The micro-controller unit 220, the feature extraction unit 230, and the switch unit 240 associate with the eye movement sensor 100 to be a system by the technology of micro physiology wireless transmitter and sensing simultaneous with analysis. The eye movement sensor 100 is used for capturing an eye movement signal of the person and converting it into an electrooculography, as said before. The micro-controller unit 220 is used for processing the electrooculography from the eye movement sensor 100. The feature extraction unit 230 is used for analyzing the electrooculography from the micro-controller unit 220 and sending it back to the micro-controller unit 220. The switch unit 240 is used for deciding the analysis result of the electrooculography from the micro-controller 220 unit and sending a deciding result back to the micro-controller unit 220. Furthermore, the micro-controller unit 220 reads automatically the electrooculography which will be calculated to obtain the sympathetic function by various operating formula. The frequency range of the electrooculography can be limited within 0.01-5 Hz, which represents sympathetic function, while a better frequency range of the electrooculography is within 0.05-0.5 Hz. The switch unit 240 sets up a threshold value based on every feature of the electrooculography from the feature extraction unit 230. The switch unit 240 will request the person to reduce eye movement when the feature of the electrooculography exceeds the threshold value. The switch unit 240 will request the person to increase eye movement when the feature of the electrooculography is below the threshold value.

Referring to FIG. 3, it shows a process flow chart of the eye movement detector 150 of the eye movement sensor 100 according to the present invention. U.S. Pat. No. 7,277,746 “Methods and Apparatus for Analyzing Heart Rate Variability”, TW 363404 “electrocardiogram signal converter for Analyzing Heart Rate Variability”, and TW 225394 “Methods and Apparatus for Analyzing Heart Rate Variability” issued to “Kuo, Terry B. J.”, etc. are all cited as the reference in this invention. The eye movement detector 150 comprises a signal amplifier 320, a filter 330, an analog-to-digital converter 340, and a digital input/output device 350. The signal amplifier 320 is used for amplifying the eye movement signal. The filter 330 is used for filtering the eye movement signal. The analog-to-digital converter 340 digitizes the eye movement signal. And the digital input/output device 350 is connected to the analog-to-digital converter as a communication interface of the eye movement signal. The eye movement detector 150 also utilizes the technology of micro physiology wireless transmitter and sensing simultaneous with analysis to convert the eye movement signal to an electrooculography. Fourier transform is adopted in spectrum analysis. In the first place, any linear drift of signal is eliminated to evade the interference from low-frequency band, and the Hamming computation is employed to prevent the mutual leakage between individual frequency components of the spectrum. After that, 288-second data (2048 points) is taken and fast Fourier transform is conducted so as to acquire heart rate power spectral density (HPSD), and the compensation with regard to any effects of sampling and Hamming computation is performed.

The micro-controller unit 220 will execute a program to process a series of analysis and control-related tasks. The digital input/output device 350 functions as the transmission interface between the micro-controller unit 220 and the subject. Transmission lines connect the signal amplifier 320 and the analog-to-digital converter 340, the analog-to-digital converter 340 and the micro-controller unit 220, and the micro-controller unit 220 and the digital input/output device 350 to transmit signals. It has to be noticed that the signal amplifier 320 and the filter 330 used in the eye movement sensor 100 are one of differential amplifier and single-ended digital amplifier.

The types of the electronic components of the device 100 for evaluating sympathetic function using electrooculography which can be implemented include: Bipolar Junction Transistor (BJT), Heterojunction Bipolar Transistor (HBT), High Electronic Mobility Transistor (HEMT), Pseudomorphic HEMT (PHEMT), Complementary Metal Oxide Semiconductor Filed Effect Transistor (CMOS) and Laterally Diffused Metal Oxide Semiconductor Filed Effect Transistor (LDMOS). Semiconductor materials broadly applicable to the electronic components of the device 100 for evaluating sympathetic function using electrooculography include: silicon, silicon-on-insulator (SOI), silicon-germanium (SiGe), gallium arsenide (GaAs), indium phosphide (InP) and silicon-germanium-carbon (SiGe—C).

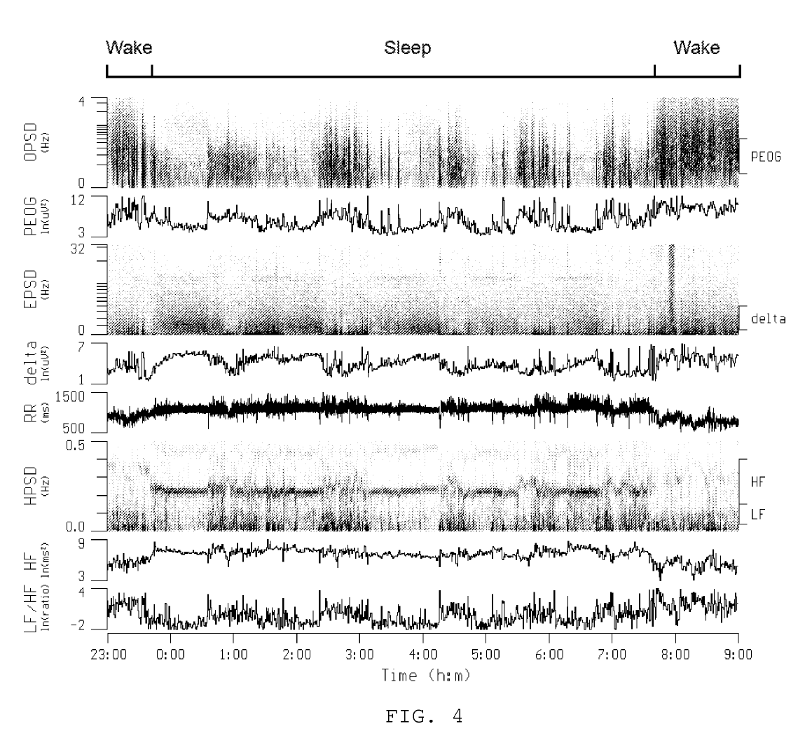

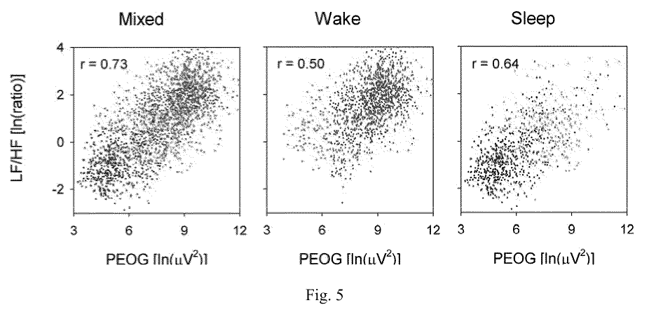

FIG. 4 and FIG. 5 show the analytical results of the eye movement frequency (0.05-0.5 Hz) intensity (PEOG) and the sympathetic function of heart (LF/HF) of the device 100 for evaluating sympathetic function using electrooculography according to the present invention. The high frequency (HF) and total power (TP) are the indexes of the heart parasympathetic function while the low frequency (LF) is the interconnected index of sympathetic and parasympathetic function, and LF/HF represents the heart sympathetic function. FIG. 4 is a recording process of a young man who is from wake through sleep to wake, which is a diagram of the eye movement frequency (0.05-0.5 Hz) intensity (PEOG) and the sympathetic function of heart (LF/HF). From the diagram, it can be found that the eye movement frequency intensity is coincident with the sympathetic function of heart. FIG. 5 is an analytical result of the eye movement frequency (0.05-0.5 Hz) intensity (PEOG) and the sympathetic function of heart (LF/HF), which includes 24 hours (mixed), wake, and sleep. From the above results, we can know that the eye movement frequency intensity within 0.05-0.5 Hz is associated to the sympathetic function of heart (LF/HF), and this appearance suits for male and female.

In conclusion, the device 100 for evaluating sympathetic function using electrooculography according to the present invention can be used in the estimation of cardiovascular disease, career, and sleep disorder of health people or various patients, or in evaluating the therapeutic effect of the long term hypnotic, evaluating the side effects of various medicines on sleep and autonomic nervous system, the change of sleep and autonomic nervous system by various health care, the change of sleep and autonomic nervous system by taking various health food, evaluating the body condition of the old man, and evaluating the sleeping problems of the babies.

Although the invention has been explained in relation to its preferred embodiment, it is not used to limit the invention. It is to be understood that many other possible modifications and variations can be made by those skilled in the art without departing from the spirit and scope of the invention as hereinafter claimed.

1. A device for evaluating sympathetic function using electrooculography comprises:
a plurality of electrode pastes, placed at the tail of each eye, and the mastoid or earlobe of the left ear and right ear of a person; and
an eye movement detector, used for capturing an eye movement signal of the person and converting it into an electrooculography.

2. The device for evaluating sympathetic function using electrooculography as claimed in claim 1, wherein the structure of the device is further selected from the group consisting of necklace, hat block, adhesive bandages, and button.

3. The device for evaluating sympathetic function using electrooculography as claimed in claim 1, wherein the eye movement detector comprises:
a signal amplifier, used for amplifying the eye movement signal;
a filter, used for filtering the eye movement signal;
an analog-to-digital converter, digitizing the eye movement signal; and
a digital input/output device, connected to the analog-to-digital converter as a communication interface of the eye movement signal.

4. The device for evaluating sympathetic function using electrooculography as claimed in claim 3, wherein the signal amplifier and the filter are one of differential amplifier and single-ended digital amplifier.

5. The device for evaluating sympathetic function using electrooculography as claimed in claim 1, wherein the frequency range of the electrooculography is within 0.05-0.5 Hz.

6. A method for evaluating sympathetic function using electrooculography mainly comprises the following steps of:
capturing an eye movement signal of a person;
performing an analog-to-digital conversion of the eye movement signal after amplifying;
converting the eye movement signal into an electrooculography; and
quantifying the electrooculography and setting up a threshold value as a basis of feedback controlling.

7. The method for evaluating sympathetic function using electrooculography as claimed in claim 6, wherein requesting the person to reduce eye movement when the feature of the electrooculography exceeds the threshold value.

8. The method for evaluating sympathetic function using electrooculography as claimed in claim 6, wherein requesting the person to increase eye movement when the feature of the electrooculography is below the threshold value.

9. A system for evaluating sympathetic function using electrooculography comprises:
an eye movement sensor, used for capturing an eye movement signal of the person and converting it into an electrooculography;
a micro-controller unit, used for processing the electrooculography from the eye movement sensor;
a feature extraction unit, used for analyzing the electrooculography from the micro-controller unit and sending it back to the micro-controller unit.

10. The system for evaluating sympathetic function using electrooculography as claimed in claim 9, wherein the structure of the eye movement sensor is further selected from the group consisting of necklace, hat block, adhesive bandages, and button.

11. The system for evaluating sympathetic function using electrooculography as claimed in claim 9, wherein the eye movement sensor comprises:
a plurality of electrode pastes, placed at the tail of each eye, and the mastoid or earlobe of the left ear and right ear of a person; and
an eye movement detector, used for capturing an eye movement signal of the person and converting it into an electrooculography.

12. The system for evaluating sympathetic function using electrooculography as claimed in claim 9, wherein the eye movement detector comprises:
a signal amplifier, used for amplifying the eye movement signal;
a filter, used for filtering the eye movement signal;
an analog-to-digital converter, digitizing the eye movement signal; and
a digital input/output device, connected to the analog-to-digital converter as a communication interface of the eye movement signal.

13. The system for evaluating sympathetic function using electrooculography as claimed in claim 12, wherein the signal amplifier and the filter are one of differential amplifier and single-ended digital amplifier.

14. The system for evaluating sympathetic function using electrooculography as claimed in claim 9, wherein the micro-controller unit reads automatically the electrooculography which will be calculated to obtain the sympathetic function by various operating formula.

15. The system for evaluating sympathetic function using electrooculography as claimed in claim 9, wherein the frequency range of the electrooculography is within 0.05-0.5 Hz.

16. The system for evaluating sympathetic function using electrooculography as claimed in claim 9, wherein the system further comprises a switch unit used for deciding the analysis result of the electrooculography from the micro-controller unit and sending a deciding result back to the micro-controller unit.

17. The system for evaluating sympathetic function using electrooculography as claimed in claim 16, wherein the switch unit sets up a threshold value based on every feature of the electrooculography from the feature extraction unit.

18. The system for evaluating sympathetic function using electrooculography as claimed in claim 17, wherein the switch unit will request the person to reduce eye movement when the feature of the electrooculography exceeds the threshold value.

19. The system for evaluating sympathetic function using electrooculography as claimed in claim 17, wherein the switch unit will request the person to increase eye movement when the feature of the electrooculography is below the threshold value.
